(19) United States
(12) Patent Application Publication
(10) Pub. No.: US 2006/0051391 A1
(43) Pub. Date: Mar. 9, 2006

(54) DEVICE FOR THE CONTROLLED ADMINISTRATION OF SUBSTANCES TO BE INSERTED IN A BODY CAVITY

(76) Inventors: Victor Oscar Dvoskin, Pcia. de Buenos Aires (AR); Julio Eduardo Massara, Pcia. de Buenos Aires (AR); Nestor Gerardo Massara, Pcia. de San Luis (AR)

Correspondence Address: ZARLEY LAW FIRM P.L.C., CAPITAL SQUARE, 400 LOCUST, SUITE 200, DES MOINES, IA, 50309-2350, US

(21) Appl. No.: 11/162,371

(22) Filed: Sep. 8, 2005

(30) Foreign Application Priority Data
Sep. 9, 2004 (AR) P 04 01 03241

(51) Int. Cl. A61F 13/00; A61K 9/22

(52) U.S. Cl. 424/422; 604/890.1

(57) ABSTRACT

Device for the controlled administration of substances to be inserted in a body cavity, characterized by: an anchoring medium allowing for a firm anchorage of the device in said body cavity; at least one substance support medium for the release of at least one substance into the body cavity following a specific protocol and in which each substance to be administered comprises an independent protocol that establishes a release kinetics with one or more substance administration stages, being each stage defined by a different release kinetics. These devices are useful to develop a simple or complex treatment protocol in mammals. The formulations applicable to the substance support media as well as the appropriate manufacturing processes are also provided.

### FIELD OF THE INVENTION

One object of the present invention is a controlled-release device, useful in the administration of active agents or combinations of simple or complex molecules in an animal cavity.

A further object of the present invention includes novel elastomeric compositions which are particularly useful in the manufacturing of the device.

A still further object of the present invention comprises the procedure to manufacture the device.

### BACKGROUND INVENTION

The transvaginal tract has enabled to develop products for the administration of active compounds through sponges with hormones, devices of the CIDR and CIDR-B lines (Ruakura Agricultural Research Centre and the Agricultural Division of Carter Holt Harvey Plastic Products Group Ltd., New Zealand, American Patent U.S. Pat. No. 6,423,039 B1), PRID (available at Sanofi Animal Health Limited, England), CUE MATE (Duirs NZ Limited, New Zealand, Australian Patent 734838) and TRIU-B (U.S. Pat. No. 6,805,877 B2). The latter is property of the same inventors. Although all of them are based on silicone rubber with the inclusion of natural progesterone, they differ in the release mechanism in vitro and in vivo.

They also differ in the manufacturing process, type of curing, area of release (internal and external) and the possibility of including disinfectants to control the median region of the vagina, in order to prevent vaginitis as well as the spreading of bacterial, parasitical and viral diseases through this tract, etc.

All the intravaginal devices with polymeric bases available in the market include natural progesterone; in the case of CIDR 1.90 g, decreasing to 1.33 grams when removed on Day 7, and to 1.05 g when removed on Day 12. Instead, PRID contains 1.55 g of progesterone at the beginning, which decreases to 1.18 and 0.94 g after 7 and 12 days respectively. In the case of PRID device, progesterone leaching can be affected by calcium salts. In the case of TRIU-B device containing 1.08 g of progesterone after 7 days and 13 days, the content decreases to 0.645 g and 0.500 g on average respectively. It is observed that with 1.080 g and 0.500 mg at 0.5 hours, it is possible to reach a level of 3.8 ng per ml in blood in the first case and 3 ng per ml in blood in the second case.

All the devices available have been designed for cattle estrus control, due to its economic importance for a profitable beef and dairy production, with implantation times for periods not exceeding 15 days.

The CIDR and TRIU-B lines' devices, which are not compliant with the Higuchi equation, produce a controlled release during a period of 7 to 8 days for the CIDR and 8 to 9 days for the TRIU-B.

Document U.S. Pat. No. 6,423,039 B1 claims an intravaginal device that can be implanted during periods covering 7 to 12 days and that includes a silicone matrix impregnated with 1 to 1.5 g of progesterone which allows to maintain a minimum of 2 ng/ml of plasma progesterone during at least 7 days. One disadvantage of this device is that it can only be used to control ovulation, since it is only useful for progesterone administration and has a release kinetics that cannot be modified. On the other hand, it cannot be implanted for more than 12 days since it can cause vaginitis due to damage to the vaginal mucous.

Document AU 734838 describes a substance release device consisting of a frame with two or more flexible arms, each having a substance support medium that can be different. However, in this case, and in order to achieve different release kinetics for each of these substances, it is necessary to remove the device, load it and then reinsert it whenever the presence of a new substance is required.

A well-known disadvantage when using the existing devices is that to achieve an effective treatment, apart from their insertion for different periods in the animal, they require injectable drugs before and/or after the implantation. These problems result in herd management difficulties and, consequently, higher costs. These techniques are almost inapplicable in species prone to stress caused by manipulation, such as gazelles and wild exotic animals.

According to the above-mentioned, the development of devices that allow for the release of one or more substances with controlled kinetics, that can be implanted for long periods or indefinitely and that enable to include a complete treatment or the equivalent to several applications of one or more drugs would simplify the handling of the animals, decrease their stress levels as well management costs.

### SUMMARY OF THE INVENTION

This is a description of a controlled release device called TDC (acronym that stands for Tratamientos Dirigidos Cavitarios [Cavity Directed Treatments]) to be used in mammals, and useful for the administration of active agents, and combinations of simple or complex molecules. These substances can pass to the systemic tract crossing the vaginal wall or not, depending on the desired place of the action, and can generate effects that will allow to control, ameliorate or cure a wide range of diseases, syndromes or physiologic conditions, some of which are included below as examples only and without any limitation whatsoever:

Reproductive Field:

To control the ovulations of different animal types or species.

To speed-up the genetic manipulation through artificial insemination at a large scale.

To improve estrus fertility of domestic, wild and exotic species.

To improve the results of embryonic transplants.

To improve the quality and quantity of embryo production of super-ovulated and cloned animals.

To contribute to the efficiency of the super-ovulation treatments or embryo transplants themselves.

To relate treatments which allow to increase the potential of embryonic retention and sustain pregnancy.

Management of Livestock Production Field:

To improve management patterns through synchronism, by organizing the calving—first estrus period of groups of animals.

To improve the nutritional management.

To improve management of the health care calendar and strengthen the active and passive immune response.

To generate treatments which allow to break the anestrus and improve estrus fertility at the new beginning of the cycles.

To increase the animal welfare potential.

To order similar metabolic situations in the recovery transition period of the cow from the post-calving period to the first estrus.

Contraception Field:

To produce contraception in the case of animals that due to their age and handling, it is not convenient for them to become pregnant.

To cancel the cycle in training animals.

Therapeutic Field:

Anti-parasite line: the novel idea is that it is possible to leach drugs such as ivermectin and many other parasiticides that allow to control endoparasites and ectoparasites in bovine, ovine, caprine, equine, porcine, dog and cat female mammals and others, offering a long action thanks to a novel pharmacokinetic profile.

Bovines. Endoparasites: gastrointestinal and pulmonary nematodes, both in immature and adult stages, including inhibited forms. Ectoparasites: acarus of the psoroptic mange, suctorial louse, “bicheras” (worms produced by flies) by Cochliomyia hominivorax, “ura”, horn fly and bovine tick.

Ovines. Endoparasites: gastrointestinal and pulmonary nematodes, both in immature and adult stages. Ectoparasites: Oestrus ovis, Melophagus ovinus, acarus of the psoroptic mange, suctorial louse, “bicheras”.

Porcines, caprines, equines. Specific parasites.

Alternatively, this device can be used in any other animal cavity, such as rumen, cul-de-sac, subcutaneous cavities, intraperitoneal cavities, etc.

### SHORT DESCRIPTION OF THE FIGURES

FIG. 1 is a perspective view of a preferred embodiment of the device of the present invention.

FIG. 2 is a top view of a preferred embodiment of the device of the present invention that includes different layers and their combinations.

FIG. 3 is a curve of progesterone plasma levels corresponding to a TDC device with 4 single-layer substance release media containing 225 mg progesterone each, made of silicone cured with cumyl peroxide, inserted for 25 days in ovariectomized cows.

FIG. 4 is a curve of progesterone plasma levels corresponding to a TDC device with 4 single-layer substance release media containing 162 mg progesterone each, made of silicone cured with cumyl peroxide, inserted in ovariectomized cows.

FIG. 5 is a curve of plasma levels corresponding to a TDC device with 4 single-layer substance release media made of silicone cured with cumyl peroxide, containing 112 mg progesterone each, and to which 3 sheaths of the same material were added, inserted in ovariectomized cows.

### DETAILED DESCRIPTION OF THE INVENTION

In a preferred embodiment of the invention, the device for the controlled administration of substances to be inserted in a body cavity includes: an anchoring medium allowing for a firm anchorage of said device in said body cavity; at least one substance support medium for the release of at least one substance into the body cavity following a pre-determined protocol; and in which each substance to be administered comprises an independent protocol that establishes a release kinetics with one or more substance administration stages, being each stage defined by a different release kinetics.

In another preferred embodiment of the invention, the protocols of each substance can be combined so as to create a more complex treatment involving the administration of two or more substances.

In still another embodiment of the invention, the said substance support medium consists of one or more layers whose composition and thickness is selected according to the desired release time and kinetics.

In a preferred embodiment, the substance release kinetics is controlled by means of adjoining layers by covering an internal layer containing the substance to be released into the body cavity with an external degradable layer in such a way that the substance contained in said internal layer is not released into the body cavity until said adjoining external layer is degraded.

In a preferred embodiment of the invention, in adjoining layers, the external layer covers the internal layer completely so that the release of the substance contained in the internal layer begins simultaneously in the entire layer.

In a preferred embodiment, in adjoining layers, the external layer covers the internal layer partially so that the release of the substance contained in the internal layer begins at different times as the latter is exposed to the medium.

In still another preferred embodiment, said external layer is inert and does not contain a substance to be released into the body cavity.

In any of the abovementioned cases, said external layer may contain at least one substance to be released into the body cavity.

In another preferred embodiment, the substance release kinetics is controlled by selecting a layer composition that allows the substance to be spread into the body cavity without degradation of said layer.

In a preferred embodiment, the substance release kinetics is controlled by means of adjoining layers by covering an internal layer containing a substance to be released into the body cavity with an inert and non-degradable external layer so that the substance contained in said internal layer is released into the body cavity by diffusion through said external layer.

In another preferred embodiment, the substance release kinetics is controlled by means of adjoining layers by covering an internal layer containing a substance to be released into the body cavity with an inert and non-degradable external layer so that the release of the substance contained in said internal layer starts only when the non-degradable external layer is manually removed. In this case, in order to take out the non-degradable external layer, it is necessary to remove the device from the body cavity.

In still another preferred embodiment, once the substance contained in one layer has been completely released into the body cavity, a new external layer containing a new substance to be released is placed on said layer. In order to place the new external layer, the device in the body cavity has to be removed.

In order to increase the contact surface, the first external layer may have slots, trunk-conic and fusiform flat strips, longitudinal strips or fungiform cups or other shapes to increase its surface.

In the preferred embodiments of the device, the composition of the layers of said substance support medium consists of a material selected from a group of polymers, carbohydrates and hydrocarbons; in which the first ones may be elastomeric polymers that can be selected from a group consisting of silicone, polyisoprene and halo polyisobutylene.

The device of one of the preferred embodiments includes a medium to remove the device from said cavity.

The size depends on the cavity it is to be inserted into and on the species to be treated.

Preferably, the device of the present invention is used in the vagina of any kind of mammals, bovines, sheep, caprines, pigs, dogs, cats, gazelles, exotic animals, camels or female buffalos. Its use consists in inserting the device in the vagina, keeping it there for a specified time period and then removing it.

In a preferred embodiment of the device of the present invention, the substance is selected from a group formed by hypothalamus hormones: GnRh (gonadotropin-releasing hormone) and all its synthetic analogous substances, hypophysis hormone, pytuitary hormone, FSH (Follicle Stimulant Hormone), LH (Luteinizing Hormone), prolactins, oxytocins, melatonins, gonadal hormones: estrogens and its different salts and synthetic derivatives, natural progesterone, analogous substances and synthetic derivatives: norgestrel, levonorgestrel, altrenogest, medroxyprogesterone, fluorogestone and derivatives, androsterone and its derivatives, testosterone, inhibins, luteolytic hormones: prostaglandin f2 alpha and its synthetic analogous substances, anti-luteolytic hormones: prostaglandin E, uterine and placental hormones: ECG (equine chorionic gonadotropin), HCG (Human corionic gonadotropin), metabolic hormones, STH (somatotrophin), adrenocortical trophins, thyroxins, insulins, angiotensins, vasopressins and all the biochemical components and structures: neuropeptides, peptides, polypeptides, aminoacids, alpha and beta glucoproteins, aminoacids, essential aminoacids, minerals, fat-soluble and water-soluble vitamins, derivatives of Vitamin B-Complex (Choline), endoparasiticides and ectoparasiticides, endectosides such as fenbendazole molecules, albendazole sulfoxide, triclabendazole, closantel, niclosamides, ivermectins, all types and classes of antigens, antibiotics, serum and physiological, Ringer, glucose, lactose and mineral solutions, enzymes, anabolics, other hormones and any combination of them according to their action in the glands, cell receptor tissues, the dosage of which shall indicate the control of the treatment expressed in international units, Dalton, micrograms, milligrams, grams with minimized or exaggerated amounts according to the quality and quantity of the substance and the type of polymer used to control the passage of the leached substance.

In a preferred embodiment of the present invention, the substance support medium is made of a composition consisting of:

| | | |
|---|---|---|
| Polyisoprene | 100 | parts |
| Curing agent (the already mentioned types of peroxide | 0 to 8 | parts |
| or sulfur) | | |
| Primary accelerator of the guanidin or tiazole type | 0 to 4 | parts |
| Secondary accelerator of the thiuram o sulfenamide type | 0 to 3 | parts |
| Curing activator of the metallic oxide type | 0 to 8 | parts |
| Silica type loads with a high specific surface | 0 to 80 | parts |
| Metallic oxide type loads | 0 to 40 | parts |
| Colorant | 0 to 2 | parts |
| Agent of the process (metallic salts and fatty acid | 0 to 15 | parts |
| esters, organsilicones) | | |
| Active substance | 0 to 15 | parts. |

In another preferred embodiment of the present invention, the substance support medium is made of a composition consisting of:

| | | |
|---|---|---|
| Silicone elastomer (any of the types already | 100 | parts |
| mentioned) | | |
| Peroxide (any of the types already mentioned) | 0 to 5 | parts |
| Colorant | 0 to 1 | parts |
| Silica type loads with high a specific surface | 0 to 80 | parts |
| Active substance | 0.2 to 15 | parts |

In both cases, the curing process can be done by cold or heat treatment at different temperatures, thus allowing for the inclusion of thermolabile molecules.

Another preferred aspect of the present invention includes a Procedure to manufacture a substance support medium, as follows:

Mix the components according to the proportion established in the COMPOUND formulation in agreement with claims 25 or 26 in a roll mill or an inner mixer of the “Banbury”, “Kneader” or “Blades” type. The mixture must then be subject to shearing to get its complete plasticization and homogenization. The sheets of the homogenous material formed must be recovered and stored. Then, mold by injection, injection-transfer, compression or casting a specific amount of the material obtained in c). The composition of the material includes the amounts of silicone and the active agent corresponding to the final device. The mold must then be kept at a temperature between 20° C. and 230° C. during periods from 1 to 30 minutes until the end of the curing process. Optionally, the device can be removed from the mold and post-cured in a furnace at 100° C.-190° C. during 4-10 hours. The device must be recovered and packed in a protected and inviolable packaging.

In a preferred embodiment of the invention, the device also has a central structure made of a polyamide mixture (“nylon”) consisting of 20 to 90% Nylon 66 Zytel AST801 HS NC 010 or Zytel 408 NC 010, and 10 to 80% Zytel 101 L NC 010; four flexible branches project from said central structure. Two branches are longitudinal and the other two are transversal, the latter can be folded both forwards and backwards from any position they are occupying inside the cavity. The said transversal branches form an angle between 90 and 50 degrees with the longitudinal branches and the union between the transversal and longitudinal branches is made through a curved portion. The bend radius of the curve portion is between 1 and 30 mm. The size of the central structure is: 8-50 mm in length, 5-30 mm in width and 1-20 mm in thickness; and the size of the branches is 5-90 mm long. The distance between the ends of both transversal branches, measured perpendicularly to the longitudinal axis, is 40-170 mm and the distance between the ends of both longitudinal branches, measured parallel to the longitudinal axis, is 20-200 mm. Each flexible branch has a substance support medium and has holding media to fix the substance support to said branches. The abovementioned holding media consists of a thickening along the branch complementary to reductions in the diameter of a hole present in the substance support medium. The substance supports have the shape of a cylinder flattened in one of its sides, between 5-70 mm in length and a major diameter of 10-30 mm and a minor diameter of 5-20 mm, and have longitudinal slots, strips or splines on its surface. The device comprises a medium to remove it from said cavity, with a size of 40-180 mm long and a diameter of 0.5-4 mm.

The substance support medium of the device is made of a composition consisting of:

| | | |
|---|---|---|
| Silicone elastomer (any of the types already | 100 | parts |
| mentioned) | | |
| Cumyl peroxide | 0 to 5 | parts |
| Colorant | 0 to 1 | parts |
| Silica type loads with a high specific surface | 0 to 80 | parts |
| Progesterone and/or altrenogest | 0.2 to 15 | parts |

One preferred embodiment of the device contains 50-300 mg of progesterone, 1-50 mg of altrenogest, or a combination of both, and is used in the vaginal cavity of goats or sheep.

Another preferred embodiment of the device contains 1-999 mg of progesterone, 1-250 mg of altrenogest, or a combination of both, and is used in the vaginal cavity of cows.

One preferred use of the present invention consists in inserting the device in the animal's vagina, keeping it there for at least 5 days and then removing it.

The devices have variable geometry, including a central structure made of a polyamide mixture (“nylon”) consisting of 20 to 90% Nylon 66 Zytel AST801 HS NC 010 or Zytel 408 NC 010, and 10 to 80% Zytel 101 L NC 010, and have at least one branch that projects from said central structure and at least one substance support medium that is placed at the branches and is independently impregnated with the desired active agent. These substance release media are formed by layers that may be made of carbohydrates, hydrocarbons or polymers, and may be covered either mechanically or manually with different layers made of carbohydrates, hydrocarbons or polymers that can totally or partially cover said layers. These layers will have a similar or different composition, and might include other active or adjuvant molecules.

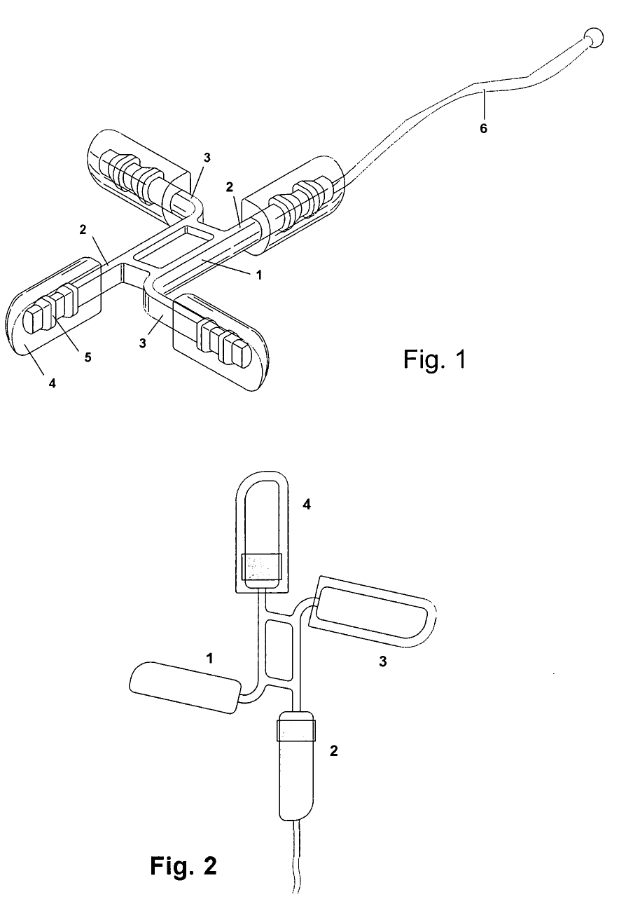

FIG. 1 shows a preferred form of the invention useful to be placed in the vagina of goats or sheep, with a cruciform central structure (1), consisting of a parallelogram with four branches, two of which are longitudinal (2) and the remaining two are transversal (3) made of the mixture of polyamides described above, which increases bending resistance. The central structure may have one or more holes. Each of these branches comprise a substance support medium (4), holding media for the latter (5) consisting of thickenings along the branch supplemented with reductions in the hole's diameter of the substance support media in which the branch is inserted with an extension or an end to facilitate its removal (6). As regards the transversal branches of the central structure (3), they are rounded and also opposite to the central structure that holds them, making up angles of 90-50 degrees with the longitudinal branches, and its bend corresponds to a circumference of 1-30 mm of radius. These characteristics enable the transversal branches to fold both forwards and backwards so they can be arranged parallel to the longitudinal branches not only during the insertion procedure but also during removal without breaking the device, and also providing a wide variety of sizes according to the species and a perfect anchorage without producing mechanical damages.

FIG. 2 shows in a single device some of the preferred embodiments of the invention: a case with one single layer (1), another case in which in the substance release media there is a second tubular layer or sheath partially covering the previous layer and allowing for the manual assembly of special devices with different compositions, depending on the desired treatment and based on layers that can be available separately (2). It also shows another preferred embodiment of the invention in which, in the substance release media, there is an external layer with a “glove” shape and which completely covers the previous layers (3). Finally, it shows a case in which the three layers are present (4). Any of these options may be found in a given device in any possible combination.

The different layers can be made of different polymeric compounds, either cured or not, depending on the substances to be included in the polymer matrix. The silicone elastomers can be of different types: HTV-High Temperature Vulcanizing (cured at high temperatures) cured with peroxides of the Dicumyl type; 2,5Dimethyl, 2,5Di(ter-buthyl hexane); Benzoil or 2,4Dichlorine Benzoil such as the LR-Liquid Rubber (liquids, cured by addition with Platinum catalyst), or RTV's-Room Temperature Vulcanizing types (cured at room temperature). If silicone elastomers are preferred, the use of types developed with other elastomeric polymers such polyisopropene, halo polyisobutilene or others is not excluded. The substances to be added as specific loads (when seeking pre-set responses of the compound) can be precipitated or pyrogenic silica, silicone polymers of low molecular weight (oils) and additives of the organosilicone type, or others. The size of the device will depend on the anatomy of the species to be treated.

Depending on the nature of the materials used, the curing process can be done by heat treatment at different temperatures or cold treatment, allowing, in the latter case, for the inclusion of proteins, glycoproteins and other thermolabile molecules that could be of interest. On the other hand, the nature of the molecules included and the release kinetics required are also considered when choosing the material, so as to enable its diffusion at the desired speed. The different substance release media of a device can be made of equal or different compounds and can produce, as a result, different times of release that surpass the ones known in the prior art. They can also release many types of drugs or molecules simultaneously, each one with a determined kinetics that will depend on the nature of the different layers selected for each case.

The use of carbohydrates as external layer allows to count on a degradable or erodable layer in the cavity so that, once eliminated, the internal layer becomes exposed and can start to leach the corresponding substance, or may contain an active substance for a quick release. With this purpose, hydrocarbons can also be used, such as paraffin or similar substances.

The wall thickness of the different layers of the substance release media is variable, according to the nature of the substance to be added and to the release kinetics desired (slow, intermediate, fast, ultra-fast).

The device of the present invention improves the anchorage in the vagina since it is lighter than the other available devices and does not damage the vaginal mucous. On the other hand, the composition of its Nylon structure allows the device to have enough memory and strength not to be expelled, broken or cracked by the contraction of the vaginal muscles of the different species to be treated. This allows its implantation for minutes, hours, weeks, or months, either continuously or intermittently, since the substance release media can be supplemented with new layers (for example gloves or sheaths) that can be manually added during the treatment.

In this type of device, the external layer's surface area of the substance release media can be modified and increased by adding slots, trunk-conic and fusiform flat strips or longitudinal strips, thus improving the versatility of the device and its contact with the vaginal membrane. In this sense, the increase in the surface area has to do with the substance release media themselves and not with the nylon structure, though both will have variable sizes according to the anatomy of the vagina of the animal to be treated.

This design enables to release as many types of drugs or molecules as substance release media are used, each one with a pre-set kinetics that will depend on their composition. It is still possible to increase this potential by adding supplementary sheaths or gloves and modifying the doses according to the species to improve their blood curves.

Release Mechanism

The release mechanism in the substance release media is a process in which the factors determining the release speed are:

a) The solubility level of the active substance in the substance support medium matrix. The substance should be soluble but to a limited extent.

This solubility depends on:
a-1) The relative polarity of the substance and the matrix.
a-2) The fusion point and the molecular size of the active substance.
a-3) The device manufacturing temperatures (mixing and molding) as compared to the usage temperature.

b) In the case of the elastomeric polymers, the density and uniformity of reticulation, which is directly related to the type of curing: by addition as in liquid silicones (higher density and ordering) or according to the type of peroxide used (lower density and relative disorder of the reticule). The addition of silicone oils can accelerate the presence of the drug and the leaching process in the vaginal tract.

c) The volume of the device (or the thickness of the walls) since the release speeds decrease when detached from the surface.

d) The contact surface with the medium.

Substance Support Medium Manufacturing Method:

The industrial operations used to transform the elastomeric materials utilized as support medium of different active principles allow for a wide range of possibilities regarding the type of mixing process and the conformation of the pieces.

The mixing process requires an open mill type machinery with a high range of friction coefficient, of the “banbury”, “kneader” or “blades” types. The temperatures used in the device molding or manufacturing processes is from room temperature to 230° C.; the “casting” system is applied at room temperature, and a vacuum device is required.

A precise composition of the elastomeric matrix and several components is defined.

A preferred embodiment consists of the following formulation:

Compound (of silicone):

| | | |
|---|---|---|
| Silicone elastomer (any of the types already | 100 | parts |
| mentioned) | | |
| Peroxide (any of the types already mentioned) | 0 to 5 | parts |
| Colorant | 0 to 1 | parts |
| Silica type loads with a high specific surface | 0 to 80 | parts |
| Active substance | 0.2 to 15 | parts |

Another preferred embodiment consists of the following formulation:

Silitec:

| | | |
|---|---|---|
| Polyisoprene | 100 | parts |
| Curing agent (the already mentioned types of peroxide | 0 to 8 | parts |
| or sulfur) | | |
| Primary accelerator of the guanidin or tiazole type | 0 to 4 | parts |
| Secondary accelerator of the thiuram o sulfenamide type | 0 to 3 | parts |
| Curing activator of the metallic oxide type | 0 to 8 | parts |
| Silica type loads with high a specific surface | 0 to 80 | parts |
| Metallic oxide type loads | 0 to 40 | parts |
| Colorant | 0 to 2 | parts |
| Agent of the process (metallic salts and fatty acid esters, | 0 to 15 | parts |
| organosilicones) | | |
| Active substance | 0 to 15 | parts |

The type and proportion of each of these components is defined when seeking a predetermined effect, such as the release speed of the active agents, the final mechanical properties of the device, the ease of manipulation and even a simpler manufacturing process. An important advantage of these formulations, compared to the prior art, is that by obtaining polymers with lower cross linking, the substance release is practically total during its use, resulting in lower quantities of residual substance than those of the prior art. The immediate consequence is the decrease in the amount of substance required in the device to obtain the same result, which leads to lower costs, given the influence of the substance cost in the final cost of the device.

The defined proportion of the components is called COMPOUND. As a univocal way of recognizing it and proving its quality stability, the curing curve called “Reometric” is used to show the passage of time in the reticulation process at a predetermined temperature.

The procedure required to manufacture the device is as follows:

a) Mix the components according to the proportion established in the COMPOUND formulation in a mixing roll mill or an inner mixer of the “Banbury”, “Kneader” or “Blades” type.

b) The mixture must then be subject to shearing to get its complete plastification and homogenization.

c) The sheets of the homogenous material formed must be recovered and stored.

d) Then, mold by injection, injection-transfer, compression or casting a specific amount of the material obtained in c). The composition of the material includes the amounts of silicone and progesterone corresponding to the final device.

e) The mold must then be kept at a temperature between 20° C. and 230° C. during periods from 1 to 30 minutes until the end of the curing process.

f) Optionally, the device can be removed from the mold and post-cured in a furnace at 100-190° C. during 4-10 hours.

g) The device must be recovered and packed in a protected and inviolable packaging.

### EXAMPLES OF THE EMBODIMENTS

The invention will be illustrated through the following examples which are not to be considered limited in scope.

Blood Level Parameters (Blood level at a stable condition): In this case, there are two levels: one is achieved by measuring the plasma profiles of ovariectomized cows, goats and sheep, and the other by applying follicular aspiration to the plasma profiles of cows, goats and sheep, where differences are observed in the curve both in CIDR Line and in TRIU and TDC Lines.

### Example 1

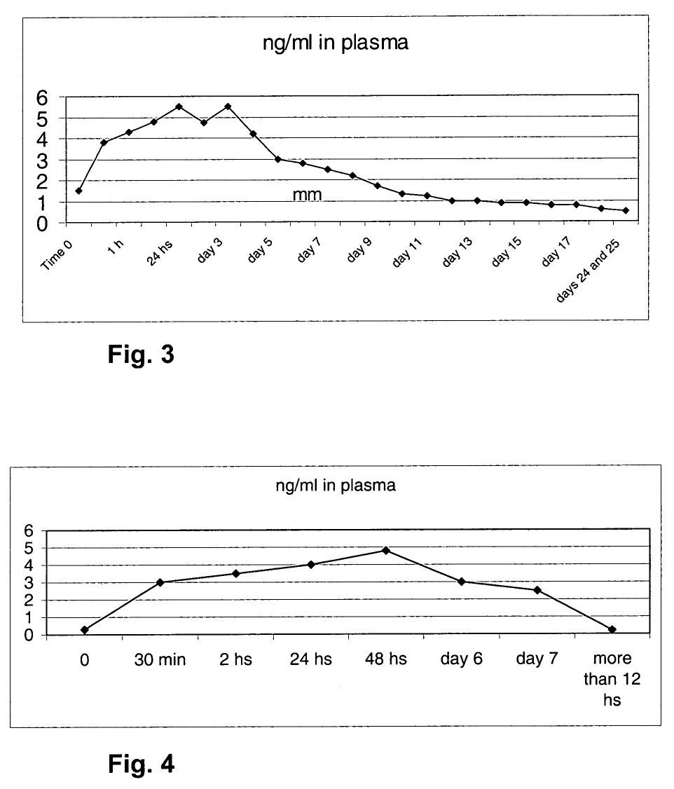

After the insertion of a TDC device (4 single-layer substance release media with 0.225 mg progesterone each, made of silicone cured with cumyl peroxide) in ovariectomized cows, the absorption phase occurs rapidly as shown in FIG. 3.

*From time 0 of TDC insertion, the p4 curve increases by 1.5 ng/ml in blood approximately every 10 minutes; the following table illustrates the values as a function of time:

| | ng/ml in |
|---|---|
| Time | plasma |
| 0 minutes | 1.5 |
| 30 minutes | 3.8 |
| 1 hour | 4.3 |
| 2 hours | 4.8 |
| 24 hours | 5.5 |
| 48 hours | 4.75 |
| Day 3 | 5.5 |
| Day 4 | 4.2 |
| Day 5 | 3.0 |
| Day 6 | 2.8 |
| Day 7 | 2.5 |
| Day 8 | 2.2 |
| Day 9 | 1.7 |
| Day 10 | 1.3 |
| Day 11 | 1.2 |
| Day 12 | 1.0 |
| Day 13 | 1.0 |
| Day 14 | 0.9 |
| Day 15 | 0.9 |
| Day 16 | 0.8 |
| Day 17 | 0.8 |
| Days 18 to 23 | 0.6 |
| Days 24 and | 0.5 |
| 25 | |

### Example 2

A TDC device with 4 single-layer substance release media with 162 mg progesterone each, made of silicone cured with cumyl peroxide was inserted in ovariectomized cows, obtaining the following levels of progesterone in plasma which are also shown in FIG. 4.

TDC- 650 mg
p4

| | Time | | | | | | | |
|---|---|---|---|---|---|---|---|---|
| | | | | | | | | More |
| | | | | | | | | than |
| | 0 | 30 min | 2 h | 24 h | 48 h | Day 6 | Day 7 | 12 hs |
| ng/ml in | 0.29 | 3 | 3.5 | 4 | 4.8 | 3 | 2.5 | 0.2 |
| plasma | | | | | | | | |

### Example 3

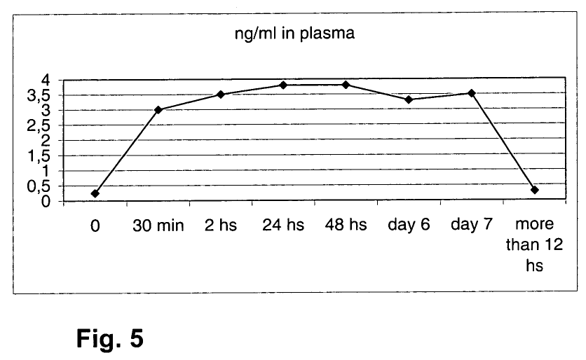

A TDC device with 4 single-layer substance release media made of silicone cured with cumyl peroxide, containing 112 mg progesterone each, and to which 3 sheaths of the same material were added, was placed continuously in ovariectomized cows. The concentrations of progesterone in plasma obtained are shown in the following table and in FIG. 5.

TDC- 450 (3 sheaths) and 380 mg (2 sheaths) of progesterone

| | Time | | | | | | | |
|---|---|---|---|---|---|---|---|---|
| | | | | | | | | More |
| | | | | | | | | than |
| | 0 | 30 min | 2 h | 24 h | 48 h | Day 6 | Day 7 | 12 h |
| ng/ml in | 0.25 | 3 | 3.5 | 3.8 | 3.8 | 3.3 | 3.5 | 0.3 |
| plasma | | | | | | | | |

The plasma concentration of these P4 curves in ovariectomized cows was compared with another placebo implant with 0 g of progesterone. Sample 1 was obtained before placing the devices, and samples 2 and 7 were taken every 2 hours during the first day (Day 0). The remaining samples were obtained daily and the sample +12 hours was taken after removal of the device every 2 hours.

With CIDR devices, blood levels reach their peak in just a few hours, whereas with TDC devices (Example 1), once placed intravaginally, in the case of the progesterone, blood levels increase by 1.5 ng/ml in blood and reach in half an hour levels between 3.8 and 4 ng/ml. The other parameters remained bioequivalent; we showed curves that remained for more than 20 days. Under the same treatment conditions, the release bioequivalence occurs for CIDR device (1.9 g of p4) against a TDC device that only contains 0.900 g of p4, apart from producing efficiency test effects of less than 2 ng/ml in blood during the 7/8/9 days after removal of the device.

This finding shows that 1 ng/ml is enough to get an efficiency equivalent to the 2 ng mentioned in document U.S. Pat. No. 6,423,039 B1.

When devices belonging to the TDC line with 650 mg of p4 (Example 2), were analyzed, p4 levels decreased proportionally by 1.5 to 2 ng/ml in blood; but in the results of the efficiency tests, there were not significant differences and, surprisingly, with p4 amounts of 450 mg (Example 3), the 1-to-7-day curves remained in a stable plateau and, 24 hours after the insertion, the curve remained at 3 ng/ml and continued to be in the same value until the end of the process. This occurred when some accessories were added (3 sheaths of 100 mg each) and, when the dosage was lowered to 380 mg, the curve was identical to the 650 mg curve. No significant differences were noted in the efficiency tests as compared to the devices containing 900 mg. The same happened with TDC devices when a device without progesterone was prepared and added an accessory attached to the device with 150 mg of medroxyprogesterone. Efficiency tests did not show significant differences with the other devices already mentioned. On the contrary, they showed a tendency to increase by over 10% the conception level in the devices with 150 mg of medroxyprogesterone and the devices with 450 mg and 380 mg of progesterone, as compared to the devices with higher amounts.

The abovementioned information indicates that the stable release condition depends on the elastomeric polymer and on the polymeric accessory used, and this is not directly proportional to the amount of progesterone because of the elastomeric polymer thickness used. Besides, and surprisingly enough, this occurs after using different breeds of animals, as can be observed in the works described.

### Example 4

The TDC (SILITEC) device containing a total amount of 150 mg of natural progesterone was used to induce and synchronize estrus in sheep and goats with a high conception rate. In this case, shorter treatment time periods can be observed, even a 5-day treatment.

However, with the same polymer and the same device of a cruciform shape, we have achieved the same results with 15 mg of medroxyprogesterone acetate, which are bioequivalent to 100 mg of natural progesterone and 6 mg of Altrenogest (derived from norgestrels).

### Example 5

When a device with four substance-release media with 125 mg progesterone each is used, the total amount on Day 7 decreases to 198. When these devices are reloaded with two sheaths of 100 mg each (total 398 mg) and 3 sheaths (total 498 mg), in both cases, curves do not exceed 3 ng/ml in blood between days 1 and 7 after the device's insertion. The most outstanding feature is that there is a 10% increase in the efficiency tests as the dosage lowers. Recent works with a 25-day duration carried out in Nelore cows have shown that TDC device curves decrease to less than 2 ng/ml in blood after Day 8, to 1.5 ng on Day 10, to 1 ng/ml on Days 11/12 and remain stable at 0.5 ng/ml up to Day 25. Surprisingly, and in line with research work by Ballas, Peña and Bo, who used a treatment with 2 mg of estradiol benzoate or GNRH (in an equivalent dosage) at the device insertion, 500 mg of prostaglandin at the device removal and 1 mg of estradiol benzoate 24 hours later, we can observe a 10% increase in the efficiency per day when TDC devices are removed on Days 8, 9 and 10.

The following examples show the usefulness of the device though they are merely illustrative:
Device TDC 1 with 450 mg of natural p4. Nine-day insertion.
Device TDC 2 with 150 mg of natural p4. Fifteen-day insertion.
Device TDC 3 with 300 mg of natural p4 in the four caps supported by the elastomeric compounds of the “Silitec” type with two complementary gloves of 100 mgr of estradiol benzoate each.
Device TDC 4 with a total amount of 900 mg. of natural p4 supported by the elastomeric compounds of the “Silitec” type with a 60-day release period.
Device TDC 5 with a total amount of 150 mg of medroxyprogesterone acetate.
Device TDC 6 with 6 mg of altrenogest.
Device TDC 7 with 6 mg of altrenogest and a complementary cold silicone sheath with 150 mgr (micrograms) of prostaglandin (racemic or isomeric) and/or a quick-release gelatin capsule of 150 mgr of GNRH or its synthetic analogous substances according to the species receiving the treatments.
   Single-Dosage Devices:
a) device for cows and female buffaloes with a total amount of 450 mg progesterone;
b) device for goats and sheep with a total amount of 150 mg progesterone containing four sheaths with 37.5 mg. Although the disinfectant will not pass through the vaginal wall, it will help control the saprophyte flora in the median region of the vagina and result in a better p4 pre-melting.

As regards the amount of substances present in these devices, it was determined that they work properly within the following ranges (according to the species or breed):

1 mg to 999 mg of natural progesterone.

2 mg to 200 mg of fluorogestone and medroxyprogesterone acetate synthetic derivatives.

1 mg to 250 mg of norgestrel derivatives, such as altrenogest.

1. Device for the controlled administration of substances to be inserted in a body cavity, characterized by:
an anchoring medium allowing for a firm anchorage of said device in said body cavity;
at least one substance support medium for the release of at least one substance into the body cavity following a pre-determined protocol; and
in which each substance to be administered comprises an independent protocol that establishes a release kinetics with one or more substance administration stages, being each stage defined by a different release kinetics.

2. The Device of claim 1 in which the protocols of each substance can be combined so as to create a more complex treatment involving the administration of two or more substances.

3. The Device of claim 1 in which said substance support medium consists of one or more layers whose composition and thickness is selected according to the desired release time and kinetics.

4. The Device of claim 3 in which the substance release kinetics is controlled by means of adjoining layers by covering an internal layer containing the substance to be released into the body cavity with an external degradable layer in such a way that the substance contained in said internal layer is not released into the body cavity until said adjoining external layer is degraded.

5. The Device of claim 4 in which, in adjoining layers, the external layer covers the internal layer completely so that the release of the substance contained in the internal layer begins simultaneously in the entire layer.

6. The Device of claim 4 in which, in adjoining layers, the external layer covers the internal layer partially so that the release of the substance contained in the internal layer begins at different times as the latter is exposed to the medium.

7. The Device of claims 4, 5 or 6 in which said external layer is inert and does not contain a substance to be released into the body cavity.

8. The Device of claims 4, 5 or 6 in which said external layer contains at least one substance to be released into the body cavity.

9. The Device of claim 3 in which the substance release kinetics is controlled by selecting a layer composition that allows the substance to diffuse into the body cavity without the degradation of said layer.

10. The Device of claim 3 in which the substance release kinetics is controlled by means of adjoining layers by covering an internal layer containing a substance to be released into the body cavity with an inert and non-degradable external layer so that the substance contained in said internal layer is released into the body cavity by diffusion through said external layer.

11. The Device of claim 3 in which the substance release kinetics is controlled by means of adjoining layers by covering an internal layer containing a substance to be released into the body cavity with an inert and non-degradable external layer so that the release of the substance contained in said internal layer starts only when the non-degradable external layer is manually removed.

12. The Device of claim 11 in which in order to take out the non-degradable external layer it is necessary to remove the device from the body cavity.

13. The Device of claim 3 in which once the substance contained in one layer has been completely released into the body cavity, a new external layer containing a new substance to be released is placed on said layer.

14. The Device of claim 13 in which in order to place the new external layer, the device in the body cavity has to be removed.

15. The Device of claim 3 in which the first external layer has slots, trunk-conic and fusiform flat strips, longitudinal strips or fungiform cups or other shapes to increase its surface.

16. The Device of any of the abovementioned Claims in which the composition of the layers of said substance support medium consists of a material selected from a group of polymers, carbohydrates and hydrocarbons.

17. The Device of claim 16 in which said polymers are elastomeric polymers.

18. The Device of claim 17 in which said elastomeric polymers can be selected from a group consisting of silicone, polyisoprene and halo polyisobutylene.

19. The Device of claim 1 in which there is a medium to remove the device from said cavity.

20. The Device of claim 1 in which the size depends on the cavity it is to be inserted into and on the species to be treated.

21. The Device of claim 1 in which the body cavity is a vagina.

22. The Device of claim 1 in which the device can be used in any kind of mammals, bovines, sheep, goats, pigs, dogs, cats, gazelles, exotic animals, camels or buffalos.

23. The Device of claim 22 in which its use consists in inserting the device in the vagina, keeping it there for a specified time period and then removing it.

24. The Device of claim 1 in which the substance is selected from a group formed by hypothalamus hormones: GnRh (gonadotropin-releasing hormone) and all its synthetic analogous substances, hypophysis hormone, pytuitary hormone, FSH (Follicle Stimulant Hormone), LH (Luteinizing Hormone), prolactins, oxytocins, melatonins, gonadal hormones: estrogens and its different salts and synthetic derivatives, natural progesterone, analogous substances and synthetic derivatives: norgestrel, levonorgestrel, altrenogest, medroxyprogesterone, fluorogestone and derivatives, androsterone and its derivatives, testosterone, inhibins, luteolytic hormones: prostaglandin f2 alpha and its synthetic analogous substances, anti-luteolytic hormones: prostaglandin E, uterine and placental hormones: ECG (equine chorionic gonadotropin), HCG (Human corionic gonadotropin), metabolic hormones, STH (somatotrophin), adrenocortical trophins, thyroxins, insulins, angiotensins, vasopressins and all the biochemical components and structures: neuropeptides, peptides, polypeptides, aminoacids, alpha and beta glucoproteins, aminoacids, essential aminoacids, minerals, fat-soluble and water-soluble vitamins, derivatives of Vitamin B-Complex (Choline), endoparasiticides and ectoparasiticides, endectosides such as fenbendazole molecules, albendazole sulfoxide, triclabendazole, closantel, niclosamides, ivermectins, all types and classes of antigens, antibiotics, serum and, physiological, Ringer, glucose, lactose and mineral solutions, enzymes, anabolics, other hormones and any combination of them according to their action in the glands, cell receptor tissues, the dosage of which shall indicate the control of the treatment expressed in international units, Dalton, micrograms, milligrams, grams with minimized or exaggerated amounts according to the quality and quantity of the substance and the type of polymer used to control the passage of the leached substance.

25. The Device of claim 1 in which the substance support medium is made of a composition consisting of:

| | | |
|---|---|---|
| Polyisoprene | 100 | parts |
| Curing agent (the already mentioned types of peroxide | 0 to 8 | parts |
| or sulfur) | | |
| Primary accelerator of the guanidin or tiazole type | 0 to 4 | parts |
| Secondary accelerator of the thiuram o sulfenamide type | 0 to 3 | parts |
| Curing activator of the metallic oxide type | 0 to 8 | parts |
| Silica type loads with a high specific surface | 0 to 80 | parts |
| Metallic oxide type loads | 0 to 40 | parts |
| Colorant | 0 to 2 | parts |
| Agent of the process (metallic salts and fatty acid esters, | 0 to 15 | parts |
| organosilicones) | | |
| Active substance | 0 to 15 | parts. |

26. The Device of claim 1 in which the substance support medium is made of a composition consisting of:

| | | |
|---|---|---|
| Silicone elastomer (any of the types already | 100 | parts |
| mentioned) | | |
| Peroxide (any of the types already mentioned) | 0 to 5 | parts |
| Colorant | 0 to 1 | parts |
| Silica type loads with a high specific surface | 0 to 80 | parts |
| Active substance | 0.2 to 15 | parts |

27. The Device of claim 25 or 26 in which the curing process can be done by heat treatment at different temperatures or cold treatment, thus allowing for the inclusion of thermolabile molecules.

28. Procedure to manufacture a substance support medium comprising the steps of:
a—mixing the components according to the proportion established in the COMPOUND formulation in claims 25 or 26 in a roll mill or an inner mixer of the “Banbury”, “Kneader” or “Blades” type;
b—shearing the mixture to get its complete plastification and homogenization;
c—recovering and storing the sheets of the homogenous material thus formed;
d—molding by injection, injection-transfer, compression or casting a specific amount of the material obtained in c), whose composition includes the amounts of silicone and the active agent corresponding to the final device;
e—keeping the mold at a temperature between 20° C. and 230° C. during periods from 1 to 30 minutes until the end of the curing process;
f—optionally, removing the device from the mold and post-curing in a furnace at 100° C.-190° C. during 4-10 hours.;
g—recovering and packing the device in a protected and inviolable packaging.

29. The Device of claims 1 or 2 in which the device also has a central structure.

30. The Device of claim 29 in which the central structure is made of a polyamide mixture (“nylon”) consisting of 20 to 90% Nylon 66 Zytel AST801 HS NC 010 or Zytel 408 NC 010 and 10 to 80% Zytel 101 L NC 010.

31. The Device of claim 29 in which the anchoring medium consists of four flexible branches projecting from said central structure.

32. The Device of claim 31 in which there are two longitudinal branches and two transversal branches, and where the transversal branches can be folded both forwards and backwards from any position they are occupying inside the cavity.

33. The Device of claim 32 in which the transversal branches form a 90-50 degrees angle with the longitudinal branches.

34. The Device of claim 32 in which the union between the transversal and longitudinal branches is made through a curve portion, being the bend radius of the curve portion between 1 and 30 mm.

35. The Device of claim 29 in which the size of said central structure is: 8-50 mm in length, 5-30 mm in width and 1-20 mm in thickness.

36. The Device of claim 32 in which the size of said branches is 5-90 mm long.

37. The Device of claim 32 in which the distance between the ends of both transversal branches, measured perpendicularly to the longitudinal axis, is 40-170 mm.

38. The Device of claim 32 in which the distance between the ends of both longitudinal branches, measured parallely to the longitudinal axis, is 20-200 mm.

39. The Device of claim 31 in which each flexible branch has a substance support medium.

40. The Device of claim 39 in which said flexible branches are the holding media to fix the substance support to said branches.

41. The Device of claim 40 in which the abovementioned holding media consist of a thickening along the branch that is complementary to reductions in the diameter of a hole present in the substance support medium.

42. The Device of claim 39 in which said substance supports have the shape of a cylinder flattened in one of its sides.

43. The Device of claim 39 in which the size of said substance supports is 5-70 mm long.

44. The Device of claim 39 in which said substance supports have a major diameter of 10-30 mm and a minor diameter of 5-20 mm.

45. The Device of claim 39 in which said substance supports have longitudinal slots, strips or splines on its surface.

46. The Device of claim 39 in which there is a medium to remove the device from said cavity, with a size of 40-180 mm long and a diameter of 0.5-4 mm.

47. The Device of any of claims 29 to 46 in which the substance has 50-300 mg of progesterone, 1-50 mg of altrenogest, or a combination of both, and is used in the vaginal cavity of goats or sheeps.

48. The Device of any of claims 29 to 46 in which the substance has 1-999 mg of progesterone and 1-250 mg of altrenogest, or a combination of both, and is used in the vaginal cavity of cows.

49. The Device of claims 47 or 48 in which its use consists in inserting the device in the animal's vagina, keeping it there for at least 5 days and then removing it.

50. The Device of claim 39 in which the substance support medium is made of a composition consisting of:

| | | |
|---|---|---|
| Silicone elastomer (any of the types mentioned) | 100 | parts |
| Cumyl peroxide | 0 to 5 | parts |
| Colorant | 0 to 1 | parts |
| Silica type loads with a high specific surface | 0 to 80 | parts |
| Progesterone and/or altrenogest | 0.2 to 15 | parts |
